(19) United States
(12) Patent Application Publication
(10) Pub. No.: US 2003/0108543 A1
(43) Pub. Date: Jun. 12, 2003

(54) Photophoretic auto immune stimulation

(76) Inventor: Leon J. Lewandowski, West Hampton, NY (US)

Correspondence Address: CRAIG G. COCHENOUR, BUCHANAN INGERSOLL, P.C., ONE OXFORD CENTRE, 20th FLOOR, 301 GRANT STREET, PITTSBURGH, PA, 15219, US

(21) Appl. No.: 10/229,708

(22) Filed: Aug. 28, 2002

Related U.S. Application Data
(60) Provisional application No. 60/339,652, filed on Dec. 12, 2001.

(51) Int. Cl. A61N 1/30; A61K 31/366; A61K 39/395

(52) U.S. Cl. 424/140.1; 514/455; 604/20

(57) ABSTRACT

A method of sensitizing a patient's immune system is disclosed. The method includes forming a reagent comprising an ultraviolet light-sensitive chemical, and optionally an antibody. The method preferably includes wherein the antibody is specific for a target cell of a patient, a target host cell of a patient or a blood-borne microbial pathogen. A composition is provided comprising an antibody and an ultraviolet light-sensitive chemical wherein the ultraviolet light-sensitive chemical is preferably a psoralen or psoralen-derived light-sensitive chemical.

### BENEFIT OF PRIOR PROVISIONAL APPLICATION

This utility patent application claims the benefit of co-pending prior U.S. Provisional Patent Application Serial No. 60/339,652, filed on Dec. 12, 2001, entitled “Photophoretic Auto Immune Stimulation” having the same named applicant as inventor, namely, Leon J. Lewandowski.

### BACKGROUND OF THE INVENTION

1. Field of the Invention

The present invention relates generally to a method of sensitizing a patient's immune system and to a composition comprising an antibody and an ultraviolet light-sensitive chemical that is employed in the method of the present invention.

02. Description of the Background Art

The immune system of a patient protects the patient's body from, such as for example but not limited to, pathogenic organisms such as bacteria and viruses. Cells and molecules of the immune system are capable of recognizing and destroying specific substances, namely immunogens. An immunogen is any substance to which an immune response can be made, usually by the production of protein molecules such as for example antibodies or immunoglobulins. An immune response in the patient occurs when a specific cell or cells of the patient recognize and react to the immunogen or immunogens. Various vaccines have been developed over time that contain a weakened or killed pathogen, such as a bacterium or virus, or a portion of the pathogens structure that upon administration to a patient stimulates antibody production against the pathogen but is generally incapable of causing severe infection. The degree of protection afforded to the patient may be quite variable. Some immune responses to vaccines or to immunogens encountered in nature may confer lifetime protection, such as for example the poliomyelitis vaccine. Other vaccines may confer only protection for a short period of time. It is postulated that the variable nature of the protection is due to the variable lifetimes of the memory cell or cells generated by the initial immunogen. Some viruses such as those causing influenza or acquired immunodeficiency syndrome (AIDS) are able to mutate their markers such that antibodies or T cell receptors responsive to the initial antigen of these pathogens are no longer effective.

In spite of this background art, there remains a very real and substantial need for a method of sensitizing a patient's immune system and for a composition having a monoclonal antibody and an ultraviolet light-sensitive chemical for use in the method of the present invention.

### SUMMARY OF THE INVENTION

The present invention has met the hereinbefore described needs. The present invention provides a method of sensitizing a patient's immune system comprising obtaining a sample quantity of a patient's blood, forming a reagent comprising an ultraviolet light-sensitive chemical, and optionally an antibody, mixing the sample of the patient's blood with the reagent to form a blood-reagent mixture, exposing the blood-reagent mixture to an ultraviolet light to establish photophoresis and the formation of a treated blood-reagent mixture, and administering the treated blood-reagent mixture to the blood stream of the patient for establishing sensitization of the patient's immune system. The method of the present invention preferably includes wherein the reagent is an ultraviolet light-sensitive chemical and an antibody that is specific for a target cell of the patient, a target host cell of the patient, or a blood borne microbial pathogen. More preferably, the method includes wherein the antibody is selected from the group consisting of a monoclonal antibody, a polyclonal antibody, and combinations thereof.

The embodiment of the present invention includes the method as described herein including employing the method as a vaccination therapy for maintaining an immune response to the immunogen or immunogens.

In another embodiment of the present invention, the method as described herein includes employing the method as therapy for the treatment of at least one blood borne microbial pathogen. Most preferably, this method includes wherein the blood borne pathogen is selected from the group consisting of bacteria, viruses, prions, and combinations thereof.

Another embodiment of the present invention provides for the method as described herein including employing the method as therapy for the control of early-stage tumor cell development.

The present invention provides a method as described herein including employing the method as therapy for the control of graft versus host and host versus graft disease.

In yet another embodiment of the present invention, the method as described herein includes employing the method as therapy for the control of aging-based generalized immune system depletion.

Another embodiment of the present invention includes a composition comprising an antibody and an ultraviolet light-sensitive chemical. In a preferred embodiment of the present invention, the composition includes wherein the ultraviolet light-sensitive chemical is a psoralen or a psoralen derived light-sensitive chemical. More preferably, the composition of the present invention includes wherein the psoralen is 8-methoxypsoralen or an analog and/or derivative thereof. Preferably, the method of the present invention includes wherein the antibody is a monoclonal antibody and/or a polyclonal antibody that is specific for a target cell of the patient, a target host cell of the patient, or a blood-borne microbial pathogen.

The method of sensitizing a patient's immune system to an immunogen or immunogens and the composition of the present invention shall be more fully understood from the following descriptions of the invention and the claims appended hereto.

### DETAILED DESCRIPTION OF THE PREFERRED EMBODIMENTS

As used herein, the term “patient” means a member of the animal kingdom including, but not limited to human beings.

The method of sensitizing a patient's immune system of the present invention is coined by the applicant as “Photophoretic Auto Immune Stimulation” (hereinafter “PAIS”) and is a novel and timely multidisciplinary mechanism to “boost” a patient's immune system into a state of improved sensitivity. The method of the present invention of sensitizing a patient's immune system comprises obtaining a sample quantity of the patient's blood, forming a reagent comprising an ultraviolet light-sensitive chemical, mixing the sample of the patient's blood with the reagent to form a blood-reagent mixture, exposing the blood-reagent mixture to an ultraviolet light to establish photophoresis and to establish the formation of a treated blood-reagent mixture, and administering the treated blood-reagent mixture to the blood stream of the patient for establishing sensitization of the patient's immune system. Preferably, the present invention includes a method of sensitizing a patient's immune system to a target immunogen or target immunogens comprising obtaining a sample quantity of the patient's blood, forming a reagent comprising an antibody and an ultraviolet light-sensitive chemical, mixing the sample of the patient's blood with the reagent to form a blood-reagent mixture, exposing the blood-reagent mixture to an ultraviolet light to establish photophoresis and to establish the formation of a treated blood-reagent mixture, and administering the treated blood-reagent mixture to the blood stream of the patient for establishing sensitization of the patient's immune system to the target immunogen or target immunogens. Preferably, the antibody is selected from the group consisting of a monoclonal antibody, a polyclonal antibody, and combinations thereof. The method of the present invention has broad-spectrum application, such as for example, but not limited to: the treatment and control of blood-borne microbial pathogens (for example, but not limited to, bacteria, viruses and prions and combinations thereof); control of early-stage tumor (cancer) cell development; control of graft versus host/host versus graft type reactions; and control of aging-based generalized immune-system depletion.

The salient thesis of PAIS is that, as one ages, one can initiate in their early stages of exposure (and then maintain) an immune response to a target immunogen or target immunogens, whether foreign or endogenous immunogen(s) which mimics the optimum natural response of an activated immune system.

For overall descriptive purposes of the applications of the method of the present invention, we exemplify the PAIS technology using the first application noted above, namely the treatment and control of blood-borne microbial pathogens; such pathogens include, but are not limited to, Human Immunodeficiency Virus (HIV), Hepatitis C Virus (HCV), the potentially-bioterrorist agents of current concern, such as for example, but not limited to, anthrax and smallpox, and any virus or microbial agent implicated in organ/tissue transplant rejection or tumor (cancer) cell development.

It will be appreciated by those persons skilled in the art that while the applicant exemplifies the methods of the present invention for the control of blood-borne microbial pathogens, it shall be understood that the methods of the present invention are equally useful as therapy for the control of early-stage tumor cell development, the control of graft versus host and host versus graft type reactions, and the control of aging-based generalized immune-system depletion all of which may or may not be related to blood-borne microbial pathogens.

Regarding the example of the treatment and control of microbial blood-borne pathogens, the key is to stimulate the patient's own immune system to control such infections naturally, thereby delaying any more aggressive classical drug therapy, as known in the art, with its proven potential for unwarranted side effects. The method (PAIS) involves withdrawing a sample of the patient's blood (for example, on average, about 250 milliliters) into a sterile disposable container where the blood, optionally may be separated centrifugally into several components, which can then be treated selectively. The method preferably includes the use of novel biological reagents, which are a combination of highly specific monoclonal and/or polyclonal antibodies (targeting a variety of specific disease-causing microbial agents, or their host cells in which these microbes live and multiply) and an ultraviolet light-sensitive chemical preferably belonging to a class of agents known by those skilled in the art as psoralens and analogs and derivatives thereof. Preferably, the psoralen is for example, but not limited to, 8-methoxypsoralen, analogs thereof and/or derivatives thereof. In the case of HIV, the target host cell is the infected T4 cell; in other microbial infections (such as anthrax) the target host cell is, for example, the infected human macrophage. Exposure of the samples to effective, controlled levels of UV light (i.e., photophoresis) causes damage/disruption of the target microbe/host cell. This, in turn, provides the equivalent of vaccine-type immunogens for auto, (i.e., self) stimulation of the immune system, once the treated sample is returned back to the patient's bloodstream.

The effects of ultraviolet (hereinafter “UV”) irradiation on cells is dependent on a combination of factors, including the chosen UV spectrum, intensity, dose and the specific techniques used to treat (process) the particular cells, tissues or organs. UV radiation generally is electromagnetic radiation with wave lengths from between about 200 nanometers (nm) and 400 nanometers. UVB radiation (290-320 nm) appears as a transitional spectrum between UVA radiation (320-400 nm) with no major cellular effects (unless combined with a photosensitizing agent, such as for example a psoralen), and UVC radiation (200-290 mn) with major immunomodulatory, cytotoxic and mutagenic effects. For example, the use of a specific monoclonal antibody combined with a photosensitizing agent creates the reagent of the present invention which then allows for the targeting of specific microbes/host cells using the most minimal (hence, the safest) level of radiation.

The use of PAIS as a safe, first-line vaccination therapy for early stage microbial infections, a process which can be repeated periodically, preserves the ultimate use of aggressive classical drug therapy (with its often substantial side effects and potential for drug resistance) for late-stage symptomatic disease. Moreover, periodic PAIS addresses the problem of periodic microbial mutation, which is the chief reason why classical type vaccines made in the laboratory often prove of little or no value with highly mutable microbes. Individualized, periodic treatment with PAIS shall become an initial treatment of choice, with minimal side effects, for the control of blood borne pathogens.

Moreover, recent events have created a new world and need for options in the war on bio-terrorism. PAIS is a therapeutic regimen designed to stimulate the individuals' own immune system, thereby controlling an infectious outbreak of any potential bio-terrorist agent and delaying the need for classical drug therapy with its often-high potential for unwanted side effects.

Two further current examples of potential microbial targets for PAIS therapy (in addition to HIV and HCV as exemplified above) are anthrax and smallpox, the former is a bacterial disease transmitted by protective spores, while the latter is viral agent transmitted by human contact.

In the case of anthrax, individuals exposed to anthrax spores are now often being treated preventively even before expressing symptoms of infection with antibiotics such as for example, the drugs ciprofloxacin or doxycycline. Antibiotics, however are only effective during the replicating (dividing/multiplying) bacterial phase and are not effective against the protective dormant spores themselves. Should any anthrax spore germinate after completion of a course of drug therapy, an active infection can initiate (or even re-initiate) and would not be detected until actual symptoms occur; at this point re-treatment with antibiotics would most likely occur. Several cycles of such spore germination, followed by antibiotic therapy, could likely lead to development of bacterial drug-resistance.

However, the use of the method of the present invention that boosts an individual's immune system shall enhance the body's ability to respond effectively to such subsequent germinations of previously dormant anthrax spores, potentially without the use of cycles of antibiotic therapy, thereby reducing the risk of developing drug resistant bacterial mutants.

Should such mutants develop, however, subsequent cycles of PAIS therapy shall be able to better control the infection by allowing the patient's activated immune system to more effectively repel the infection in its early stages.

The second potential use of PAIS-therapy against bioterrorism can be exemplified by the viral agent termed smallpox. To date, no effective antibiotic therapy is available. Treatment relies on a live virus vaccine which is questionable in both its availability and its reliability.

The use of PAIS-therapy to boost the individual's immune system to be able to effectively respond to exposure to smallpox virus therefore provides an inexpensive alternative to existing questionable vaccine therapy.

### HIV/AIDS and HCV

HIV (Human Immunodeficiency Virus)/AIDS (Acquired Immunodeficiency Syndrome) and HCV (hepatitis C virus) are global epidemics with no resolution (cure) currently available.

### HIV/AIDS (US Data) Highlights:

850,000 people living with HIV/AIDS in 1999

$2.64 billion in drugs for HIV/AIDS sold in 2000

$4004 per capita is spent in the US on HIV/AIDS healthcare

1,503,000 total number of days of care for patients with HIV/AIDS in 1998

8 days average length of stay when hospitalization required

### HCV (US Data) Highlights:

4 million Americans (vs. 200 million people worldwide) currently infected with HCV

Approx. 400,000 HIV(+) Americans co-infected with HCV

Nationwide, nearly 400,000 (20%) state prisoners currently infected with HCV

Approx. 9,000 Americans die per year from complications of HCV

HCV is the #1 reason for liver transplants in the US

Approx. total yearly cost of drug therapy for Hepatitis in the US alone exceeds $1 billion

### HIV/AIDS

The method of the present invention provides a much-needed treatment option for the approximately 100 million people worldwide that are infected with the HIV/AIDS virus and for whom there is currently no cure and only minimally effective therapies available.

Globally, the HIV/AIDS pandemic continues to sweep across continents: the number of estimated adult HIV infections worldwide has more than doubled since 1990 from 10 million to a mid-1996 total of 25.5 Composed of distinct epidemics, each with its own features and force, the pandemic is disproportionately impacting the developing world.

Moreover, HIV continues to spread in the industrialized world, where, increasingly, it affects people who, for reasons of race, sex, behavior or social and economic status, have lesser access to services. From a global perspective, the needs for effective prevention and care are escalating. But the pandemic has now become immensely complex. It has become fragmented and is now a mosaic composed of a multitude of epidemics, which can be distinguished on the basis of: predominant modes of transmission, geographic focus, HIV sub-types, age, sex, socioeconomic or behavioral characteristics of the populations most affected, and rapidity of or potential for HIV spread.

The ultimate goal of HIV treatment research is finding a cure. With the early dramatic success of potent combination therapy in suppressing viral RNA and DNA levels below the technical limits of quantification, a reasonable hope emerged that complete removal of replication-competent HIV DNA from the human body was attainable. However, it was soon obvious that halting treatment, even after sustained periods of successful suppression, resulted in viral rebound within days or weeks to pre-treatment levels.

The dynamics of these viral blooms provided an opportunity to accurately quantify the rate of viral replication. This new information along with new understanding about the cycles of T-cell replication and activation yielded insight into the long-term nature of HIV infection. It became apparent that, despite clearance from the blood, HIV remained sequestered in various compartments and latent reservoirs throughout the body. Estimates for the duration of continuous suppressive treatment required eradicating virus in these reservoirs ranged from 10 to 60 years. Strategies of flushing the reservoirs by stimulated activation with cytokines such as IL-2 have been proposed but not realized. Research began to demonstrate that the problems of resistance, tolerability, toxicity and adherence made continuous suppression unlikely. As the limits of currently available treatment regimens have become clear, a consensus is emerging that eradication, although of great interest as a subject of research, is not a practical clinical objective at this time.

The goal of eradication aside, other arguments can be made for prompt initiation of treatment, rapid viral suppression and long-term maximal adherence. It is well understood that the capacity of the immune system is impaired as HIV infection progresses and that advanced HIV disease is marked by selective losses of immunity to specific opportunistic pathogens.

Another major concern involves the prevention of viral evolution. Despite an individual's inoculation with a genetically homogenous founder strain, the rapid kinetics of HIV replication insures daily production of thousands of viral mutants, some of which may have a selective advantage in the environment of the new host. Some of these mutations may have a broader range of cellular targets or an increased ability to cause harm.

When antiviral drugs are added to the host environment, the overall replication rate of HIV is greatly slowed but not stopped. Day to day variation in blood levels of the drugs as well as limited penetration into certain cellular reservoirs can allow replication interruptions and breakouts. For a majority of people on anti-retroviral therapy, inhibited but ongoing replication eventually produces a viral mutant able to replicate despite the drugs.

Despite the apparent success of anti-retroviral therapy in the U.S., treatment failures resulting in opportunistic infection and death from advanced HIV disease are not rare. Resistance to drugs can quickly develop due to sub-optimal dosing, incomplete adherence, random selection or acquired resistant strains. There is a large subset of patients who have been on a series of drugs over time and have developed resistance to agents in early class of inhibitor. Efforts to intensify treatment with four, five or six drugs has met with marginal success and limited tolerability. This drug-therapy selection of drug resistant mutants has recently provoked a new approach to drug therapy, namely, do not initiate active drug therapy until disease symptoms (AIDS) actually appear.

Despite the evident success of current era treatments in preventing AIDS and death in most treated individuals, clinicians and patients are becoming alarmed about the increasing incidence of potentially dangerous treatment-related metabolic toxicities. The toxicities along with the difficulty in tolerating and adhering to treatment regimens have called into question the feasibility and wisdom of attempting unremitting, long-term classical drug therapy. Despite U.S. treatment guidelines that tentatively offer treatment when CD4+ cell counts fall below 500 cell/mm³, many experts are noting an emerging consensus that accepts lowering this “when to start” value to around 350 cell/mm³ or lower.

Although the immunogenic value of scheduled treatment interruption is still unclear, evidence from these studies is providing reassurance that treatment interruption can be safe if carefully monitored. Viral rebound rates seem to be predictable, drug resistance does not usually develop, and resuppression with the same regimen is often possible. On the other hand, once all treatment is removed, HIV damage of lymph node tissues resumes and CD4+ cell count gains can be rapidly lost.

Interruption remains an investigational technique. It may in any case prove useful for managing toxicity and for negotiating treatment fatigue and adherence issues with longterm responders. Other approaches to minimizing toxicity under study include the use of lipid-lowering agents, vitamin supplementation, as well as the tactical switching or avoidance of the anti-retroviral agents implicated in specific toxicity syndromes.

Other researchers have observed that virus with evolved resistance to AZT (3′azido-3′-deoxythmidine) and other nucleoside analogs may actually become hypersensitive to NNRTI (non-nucleoside reverse transcriptase inhibitors) agents even when NNRTI resistance has previously been detected. Resistance phenotype assays are becoming available that will allow more precise characterization of drug resistance and susceptibility. Many clinicians, guided by resistance assays results, now recommend only switching single drugs rather than entire regimens when viral rebound occurs. Finally, new drugs with potentially unique resistance profiles or improved potency are slowly becoming available in clinical trials. These experimental agents may be the only good options for those with broadly cross-resistant HIV.

Although HIV is able to efficiently and insidiously use the tools of the body's immune defenses as mechanisms for its propagation, the immune system retains considerable capacity to control the virus. The long duration of infection and slow decline before overt illness appears is a measure of the struggle the body is able to sustain. Only when the immune system is worn down and exhausted does the cascade of overwhelming events known as AIDS occur.

There is considerable natural variation in the susceptibility of individuals to infection and disease progression. Some few individuals who genetically lack a crucial co-receptor necessary for HIV cellular penetration appear to be nearly immune to the virus. Others seem to have genetic factors that predispose to rapid disease progression.

Complete viral suppression through drug therapy fails to employ the body's ability to generate a protective immune response. Maintaining a chronic, low-grade infection that achieves a balance between virulence and immune control may be accomplished through chronic antigenic stimulation.

Since 1987, approximately 30 experimental HIV vaccines have been tested in people and an effective one has yet to be found. Nevertheless, spending on HIV vaccine research by the NIH is projected to be $282 million in 2001, a doubling of the vaccine budget since 1997. These monies do not reflect private dollars on such vaccine research being spent by the pharmaceutical industry.

Vaccines being tested in humans utilize various approaches to mounting a strong stimulation for the human immune system to respond with a protective response to the HIV infection. These approaches include using: a) naked viral nucleic acid to provoke host cells to produce HIV proteins to stimulate both antibody and cellular base immunity; b) classical methods to stimulate antiviral antibodies in responses to HIV surfaces proteins; c) genetically engineered viruses or bacteria to carry HIV genes into host cells; and d) combinations of the above technologies.

Many researchers, however, believe that at least for now the goal of preventing infection may be out of reach. Rather, they have shifted into a strategy to develop a therapeutic vaccine that instead of preventing infection, will control it, prevent the progression of the disease, and possibly reduce a person's risk of transmitting HIV by holding down the levels of virus in the blood stream and secretions.

Part of what makes HIV so formidable is that unlike viruses such as polio and measles, HIV literally hides in the cells of the individual's immune system. While antibodies are indeed made, these antibodies are generally not successful in blocking further infection possibly because HIV can mutate so rapidly, that the immune system cannot design new antibodies fast enough to keep up.

The method of the present invention bolsters the patient's body's immune system in a manner which can contain even rapidly - mutating viruses by stimulating both normal antibodies and cellular immunity mechanisms. The added bonus of the use of the method of the present invention as a safe first line vaccination therapy is that it preserves the ultimate use of current classical antiviral drug therapy, with its often substantial side effects profile, proven potential for developing drug-resistance mutants, and recognizable high cost of treatment, for later-stage symptomatic disease.

### HCV

HCV (hepatitis C virus) is the most common blood-borne infection in the United States. Some 4 million Americans (about 1.8% of the population) are currently infected. Among African Americans this percentage is estimated to be 3.2%. Although the number of new infections dropped dramatically during the last decade, millions of Americans remain infected and at risk for fatal liver disease. HCV infection is now the number one reason for liver transplants in the U.S.

Recently, HCV has been found in a growing number (currently 16%) of overall HIV patients. In the U.S., 80-100% of HIV-positive hemophiliacs are coinfected with HCV, as a result of contaminated blood transfusions. Approximately 70% of HIV positive drug users are also co-infected with HCV. HIV patients are now being hospitalized for HCV liver disease more frequently than for classic AIDS problems. For example, physicians from Cook County hospital reported recently that 35% of all deaths in the year 2000 in HIV-positive patients were due to liver failure associated with HCV co-infection. Moreover, prison officials say that nearly 10,000 inmates in New York and thousands more across the country are infected with the hepatitis C virus. Prison and public health officials are wrestling with how to respond to the surprisingly high rates of infection to figure out how to contain its spread, and how and when to provide expensive treatment that in most cases does not work. Some states are treating hundreds of prisoners infected with hepatitis C virus while others are treating none. The method of the present invention may be employed as a therapy for the treatment of HCV.

### Prevention of Organ Tissue Transplant Rejection, Control of Early-Stage Tumor Cell Development, and Therapy for Aging-Based Generalized Immune-System Depletion

The method of the present invention not only includes utilizing select antibodies, preferably monoclonal antibodies, and ultraviolet light-sensitive chemicals for selectively targeting those cellular components of the blood which relate to specific diseases and infections caused by blood-borne microbial pathogens, but also includes utilizing select antibodies, preferably monoclonal antibodies, and ultraviolet light-sensitive chemicals for selectively targeting those cellular blood-borne components which relate to organ tissue transplant rejection, control of early-stage tumor cell development, and therapy for aging-based generalized immune-system depletion.

The method of the present invention as described herein includes wherein the antibody, preferably a monoclonal antibody, is specific for a target cell of the patient, a target host cell of the patient or a blood-borne microbial pathogen. The blood-borne microbial pathogen is, for example, bacteria, viruses, prions, and combinations thereof.

It is known to those skilled in the art that the addition of photophoretic techniques to drug-based immunosuppressive therapy has been shown to significantly decrease the risk of cardiac rejection without any increased incidence of procedure-associated infections. The mechanism by which photophoresis blunts the acute rejection response is unknown but the finding clearly suggests a broad immunomodulatory potential for the process in clinical medicine, with a positive role for these treatments on solid-organ transplantation and patient survival. The method of the present invention, as described herein is employed as therapy for the control of graft versus host and host versus graft diseases.

The method of the present invention, as described herein, is employed as therapy for the control of early-stage tumor cell development and as therapy for aging-based generalized immune system depletion.

Another embodiment of the present invention includes a composition comprising an antibody, and preferably wherein the antibody is selected from the group consisting of a monoclonal antibody, a polyclonal antibody, and combinations thereof, and an ultraviolet light-sensitive chemical.

In another embodiment of the present invention, the composition includes wherein the ultraviolet light-sensitive chemical is a psoralen, an analog of a psoralen, or a psoralen derived light sensitive chemical. Preferably, the composition of the present invention includes wherein the psoralen is 8-methoxypsoralen, and/ or analogs thereof, and/or derivatives thereof. More preferably, the composition of the present invention includes wherein the antibody is linked to the ultraviolet light-sensitive chemical. The antibody is conveniently linked to the ultraviolet light-sensitive chemical by routine procedures using commercially available chemicals known by those persons skilled in the art.

Whereas particular embodiments of this invention have been described herein for purposes of illustration, it is evident to those persons skilled in the art that numerous variations of the details of the present invention may be made without departing from the invention as defined in the appended claims that follow.

What is claimed is:

1. A method of sensitizing a patient's immune system comprising:
obtaining a sample quantity of said patient's blood;
forming a reagent comprising an ultraviolet light-sensitive chemical;
mixing said sample of said patient's blood with said reagent to form a blood-reagent mixture;
exposing said blood-reagent mixture to ultraviolet light to establish photophoresis and to establish the formation of a treated blood-reagent mixture; and
administering said treated blood-reagent mixture to the blood stream of said patient for establishing sensitization of said patient's immune system.

2. The method of claim 1 including wherein said ultraviolet light-sensitive chemical comprises a psoralen, an analog of a psoralen, or a psoralen derived light-sensitive chemical.

3. The method of claim 2 including wherein said psoralen is 8-methoxypsoralen, an analog of 8-methoxypsoralen, or derivatives thereof.

4. The method of claim 1 including exposing said blood-reagent mixture to said ultraviolet light having an UVB radiation spectrum from about 290 to 320 nanometers, an UVA radiation spectrum from about 320 to 400 nanometers, or an UVC radiation spectrum from about 200 to 290 nanometers, or combinations thereof.

5. The method of claim 1 including employing said method as therapy for the treatment of a blood-borne microbial pathogen.

6. The method of claim 5 including wherein said blood-borne microbial pathogen is selected from the group consisting of bacteria, viruses and prions.

7. The method of claim 1 including employing said method as therapy for the control of early-stage tumor cell development.

8. The method of claim 1 including employing said method as therapy for the control of graft versus host and host versus graft diseases.

9. The method of claim 1 including employing said method as therapy for the control of aging-based generalized immune system depletion.

10. A method of sensitizing a patient's immune system to a target immunogen or target immunogens comprising:
obtaining a sample quantity of said patient's blood;
forming a reagent comprising an antibody and an ultraviolet light-sensitive chemical;
mixing said sample of said patient's blood with said reagent to form a blood-reagent mixture;
exposing said blood-reagent mixture to ultraviolet light to establish photophoresis, and to establish the formation of a treated blood-reagent mixture; and
administering said treated blood-reagent mixture to the blood stream of said patient for establishing sensitization of said patient's immune system to said target immunogen or said target immunogens.

11. The method of claim 10 including wherein said antibody is selected from the group consisting of a monoclonal antibody, a polyclonal antibody, and combination thereof, specific for a target cell of said patient, a target host cell of said patient, or a blood borne microbial pathogen.

12. The method of claim 10 including wherein said ultraviolet light-sensitive chemical comprises a psoralen, an analog of a psoralen, or a psoralen derived light-sensitive chemical.

13. The method of claim 12 including wherein said psoralen is 8-methoxypsoralen, an analog of 8-methoxypsoralen, or derivatives thereof.

14. The method of claim 10 including exposing said blood-reagent mixture to said ultraviolet light having an UVB radiation spectrum from about 290 to 320 nanometers, an UVA radiation spectrum from about 320 to 400 nanometers, or an UVC radiation spectrum from about 200 to 290 nanometers, or combinations thereof.

15. The method of claim 10 including employing said method as a vaccination therapy for maintaining an immune response to said target immunogen or said target immunogens.

16. The method of claim 10 including employing said method as therapy for the treatment of a blood-borne microbial pathogen.

17. The method of claim 16 including wherein said blood-borne microbial pathogen is selected from the group consisting of bacteria, viruses and prions.

18. The method of claim 10 including employing said method as therapy for the control of early-stage tumor cell development.

19. The method of claim 10 including employing said method as therapy for the control of graft versus host and host versus graft diseases.

20. The method of claim 10 including employing said method as therapy for the control of aging-based generalized immune system depletion.

21. A composition comprising:
an antibody, and
an ultraviolet light-sensitive chemical.

22. The composition of claim 21 wherein said ultraviolet light-sensitive chemical is a psoralen, an analog of a psoralen, or a psoralen derived light sensitive chemical.

23. The composition of claim 22 wherein said psoralen is 8-methoxypsoralen, an analog of 8-methoxypsoralen, or derivatives thereof.

24. The composition of claim 21 wherein said antibody is selected from the group consisting of a monoclonal antibody, a polyclonal antibody, and combinations thereof.

25. The composition of claim 21 wherein said antibody is linked to said ultraviolet light-sensitive chemical.

26. The composition of claim 25 wherein said antibody is selected from the group of a monoclonal antibody, a polyclonal antibody, and combinations thereof.
